(19) United States
(12) Patent Application Publication
(10) Pub. No.: US 2011/0106179 A1
(43) Pub. Date: May 5, 2011

(54) Set Screw Having Variable Pitch Thread for Use With Spinal Implant Systems

(75) Inventors: Julien J. Prevost, Memphis, TN (US); James J. Mirda, Cordova, TN (US)

(73) Assignee: WARSAW ORTHOPEDIC, INC., Warsaw, IN (US)

(21) Appl. No.: 12/609,728

(22) Filed: Oct. 30, 2009

(51) Int. Cl. A61B 17/86

(52) U.S. Cl. 606/308; 606/301

(57) ABSTRACT

An improved set screw for use with spinal implants to attach an elongated member to a bone. The set screw has a body with a lower portion. An external thread is on the lower portion wherein at least a portion of the external thread has a variable pitch. The set screw will engage a receiver member by a predetermined longitudinal distance before encountering the variable pitch of the external thread on the set screw. A preselected torque value for seating the set screw is achieved at this predetermined longitudinal distance of thread engagement by the set screw into the receiver member. The variable pitch provides consistent clamping force even when excessive torque is applied to the set screw. An upper portion can be connected to the lower portion of the set screw through a frangible portion. The preselected torque value for seating the set screw is selected at a value less than the break-off or separation torque required to cause the frangible portion to separate the upper portion from the lower portion of the set screw.

### BACKGROUND

The present invention relates generally to set screws for use with spinal implants to attach an elongated member to a bone and, more particularly, to set screws with a variable pitch thread which provides consistent clamping force even when excessive torque is applied to the set screw.

Set screws are often used with spinal implants to attach an elongated member to bone such as vertebrae or some other portion of the spinal column within a patient during a surgical procedure, such as, when the spinal column is reinforced or realigned using an elongated member. Examples of spinal implants include but are not limited to multi-axial screws, fixed-angle screws, and offset screw connectors. The spinal implants may include a receiver member with a receptacle to receive the elongated member, and an anchor (e.g., screw, hook) that attaches to the bone. The spinal implants may be constructed as a single piece, or may include multiple pieces that are attached together such as a multi-axial screw that includes a receiver member that attaches to a head of a bone screw. The elongated member fits within the receptacle of the receiver member, and the set screw attaches to the receiver member to prevent escape of the elongated member. The set screw may also apply a clamping force to the elongated member to maintain the attachment within the receiver member. The spinal implants may be configured to receive a variety of different elongated members, including but not limited to rods, cables, and wires.

When the elongated member is made of a polymeric material, it can not always withstand the same amount of compressive or clamping force as a traditional elongated member made of metal. An elongated member made of polymeric material may become damaged if compressed with the same amount of force typically used to secure an elongated member made from a traditional metal material and may also become dislodged if not sufficiently tightened. Regardless of the material from which an elongated member is made, most surgeons desire to apply substantially the same torque to the set screw as they would normally apply when attaching the elongated member to the receiver member. Surgeons that have performed countless spinal surgeries may have become accustomed to a particular tactile feel in order to assure a spinal implant is securely tightened. Changes in this procedure or in torque value when using an elongated member made of a different material could result in mistakes and could lead to errors in securing the elongated members which may cause detrimental results in patient outcomes.

Accordingly, devices, systems, and methods for overcoming these shortcomings are needed.

### SUMMARY

In one aspect of the present invention, a set screw for use with a receiver to secure an elongated member in a spinal implant system is presented. The set screw has a body with an upper portion and a lower portion. The upper portion is connected to the lower portion through a frangible portion. An external thread is on the lower portion wherein at least a portion of the external thread has a variable pitch.

In another aspect of the invention, the external thread includes at least a portion of the external thread having a standard pitch and at least a portion having a variable pitch. The external thread has at least a first and second crest wherein the external thread has a variable pitch that starts after the second crest. The external thread is a reverse angle thread. The external thread includes a thread start having a truncation cut. A beginning of the truncation cut is at an angle of greater than 120° from an exit of the truncation cut. A reference datum on the upper portion and the truncation cut begins at a location aligned with the reference datum. The external thread has a variable pitch along the entire length of the thread. A hollow bore is formed in the body of the set screw and the hollow bore extends from an opening in the upper portion through the frangible portion and into at least a portion of the lower portion. The portion of the hollow bore extending into the lower portion comprises a driving recess for assisting in the removal of the lower portion after the upper portion has been broken away at the frangible portion. The frangible portion is an area of reduced wall thickness and can have a cross-section in the form of a notch creating a weakened zone. The upper portion can have at least one flat surface for the application of torque to the set screw. This flat surface can be on the exterior of the upper portion.

In yet another aspect of the invention, a spinal implant system is presented. The spinal implant system includes an elongated member, a set screw and a receiver member. The elongated member can have a polymeric outer surface. The set screw includes an upper portion connected to a lower portion by a frangible portion. The lower portion is separable from the upper portion by breaking the frangible portion. The lower portion has an external thread with a variable pitch. The receiver member has a proximal end and a distal end. The distal end is adapted to be affixed to the vertebral column of a patient during spinal surgery. The proximal end of the receiver member has a threaded opening leading to a U-shaped receptacle between a first extending arm and a second extending arm. The threaded opening is configured to engage the external thread of the set screw. The elongated member is capable of being secured within the U-shaped receptacle of the receiver member by the set screw without causing detrimental deformation of the polymeric outer surface of the elongated member. A break-off torque for separation of the frangible portion is greater than the amount of torque needed to secure the elongated member in the U-shaped receptacle of the receiver member by the set screw. The first extending arm has a first proximal face and the second extending arm has a second proximal face. The first proximal face has at least a partial thread formed therein to engage the exterior thread of the set screw. The second proximal face has wholly omitted any thread being formed therein to assure the external threads engage the first extending arm before engaging the second extending arm in order to avoid cross-threading of the exterior thread on the set screw. The external thread of the set screw includes at least a portion of the external thread having a standard pitch and at least another portion having a variable pitch. The external thread of the set screw includes a thread start having a truncation cut. The truncation cut begins at a location aligned with a reference datum on the upper portion of the set screw so that the set screw will engage the receiver member by a predetermined longitudinal distance before encountering the variable pitch of the set screw.

These and other aspects, forms, objects, features, and benefits of the present invention will become apparent from the following detailed description and drawings.

### BRIEF DESCRIPTION OF THE DRAWINGS

In the accompanying drawings, which are incorporated in and constitute a part of the specification, embodiments of the present invention are illustrated, which, together with a general description of the invention given above, and the detailed description given below, serve to exemplify the embodiments of this invention. The components in the Figures are not necessarily to scale, emphasis instead being placed upon illustrating the principles of the invention and, in the Figures, like reference numerals designate corresponding parts throughout the different views.

FIG. 1 is a perspective view of a spinal implant system according to one embodiment of the present disclosure.

FIG. 2 is a perspective view of a receiver member according to one embodiment of the present disclosure.

FIG. 3 is an elevation view of a set screw according to one embodiment of the present disclosure.

FIG. 4 is an end view of the set screw shown in FIG. 3.

FIG. 5 is a cross-sectional view of the set screw shown in FIG. 3.

FIG. 6 is a perspective view of a set screw according to another embodiment of the present disclosure.

FIG. 7 is a partial view of the cross-sectional view shown in FIG. 5.

FIG. 8 is an opposing end view of the set screw shown in FIG. 3.

FIG. 9 is a graph illustrating a plot of axial length versus pitch according to one embodiment of the present disclosure.

FIG. 10 is a graph illustrating a plot of axial length versus variable pitch according another embodiment of the present disclosure.

FIG. 11 is a graph illustrating a plot of axial length versus variable pitch according with yet another embodiment of the present disclosure.

FIG. 12 is a cross-sectional view of the spinal implant system according to another embodiment of the present disclosure.

### DETAILED DESCRIPTION

The present disclosure relates generally to the field of orthopedic surgery, and more particularly to devices, systems and methods for correction of spinal deformities through the use of set screws allowing fixation of elongated member 3 without damaging the same. For the purposes of promoting an understanding of the principles of the invention, reference will now be made to embodiments or examples illustrated in the drawings, and specific language will be used to describe these examples. It will nevertheless be understood that no limitation of the scope of the invention is thereby intended. Any alteration and further modifications in the described embodiments, and any further applications of the principles of the invention as described herein are contemplated as would normally occur to one skilled in the art to which the disclosure relates.

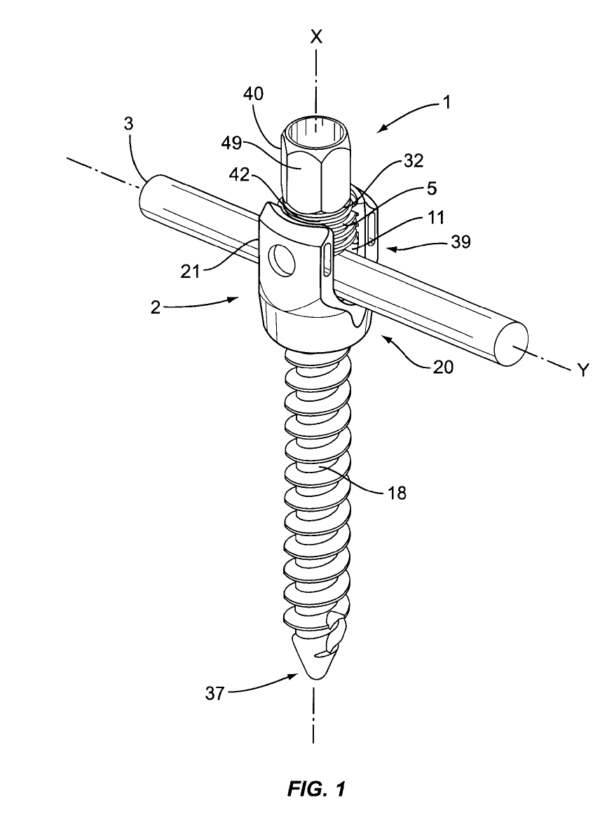

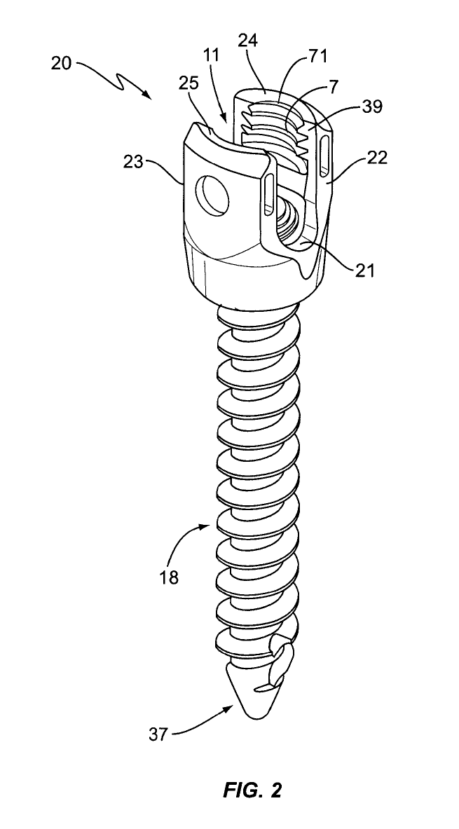

Referring to the drawings in more detail, the reference numeral 1 generally refers to a set screw 1 for use in spinal implants and in particular for use in spinal implant system 2. As shown in FIGS. 1-2, the set screw 1 is adapted for use in securing an elongated member 3 of the spinal implant system 2 relative to a receptacle 21 of a receiver member 20 to avoid translational or rotational movement. The receptacle 21 is of the type formed in the receiver member 20 of a bone screw 18 or the receiver member 20 of a connector or bone hook. In the field of spine surgery, bone screw 18 is often referred to as sacral screws or pedicle screws. The elongated member 3 may be of the type including spinal rods or the arm or rod portion of a connector. The illustrated elongated member 3 in FIG. 1 is of a round cross-section; however, it is foreseen that the elongated member 3 could be rectangular to help prevent rotation in a similarly shaped bore in the receiver member 20. Alternatively, the elongated member 3 may have a cross-section of almost any shape, including for example, an oval, hexagon, T or I-beam, triangle, or star shape. In a U-shaped receptacle 21 a threaded opening 11 leads to and typically has an longitudinal axis X substantially perpendicular to the axis Y of the elongated member 3 which passes through the U-shaped receptacle 21 and the threaded opening 11 extends radially relative to the axis Y of the elongated member 3. This is also typical for receiver member 20 that have a closed loop receptacle 21 such as in a closed hook, closed screw or closed connector (not shown). For hooks, screws and connectors that use the U-shaped receptacle 21 illustrated, the angle or point of penetration on the elongated member 3 through the receptacle 21 may vary, for example in a sagittal direction, with respect to the axis Y of the elongated member 3.

The U-shaped receptacle 21 includes a first extending arm 22 with a first proximal face 24 and a second extending arm 23 with a second proximal face 25. The first proximal face 24 has at least a partial thread 71 formed therein to engage the external thread 5 of the set screw 1. The second proximal face 25 has wholly omitted any thread being formed therein to assure the external thread 5 engage the first extending arm 22 before engaging the second extending arm 23 in order to avoid cross-threading of the external thread 5 on the set screw 1.

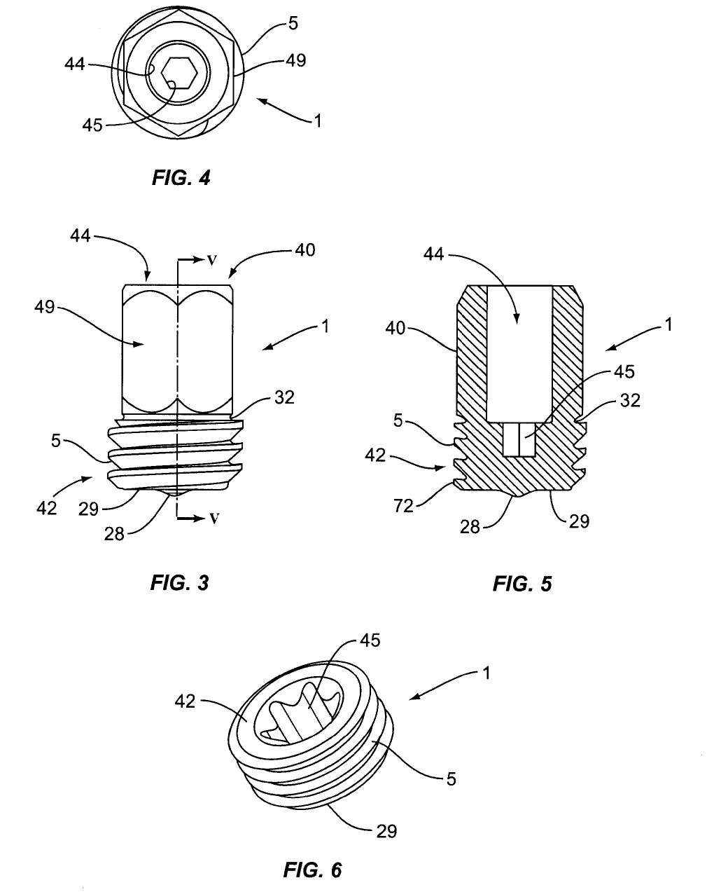

A set screw 1 may, as is shown in FIGS. 3-4, comprise a upper portion 40, of hexagonal external cross-section and round internal cross section, and a lower portion 42, having an external thread 5 on an outer circumferential surface. The upper portion 40 has a length and width that is configured to facilitate manipulation of the set screw 1. A bottom tip 28 is formed on a bottom end 29 of the set screw 1 and is located centrally on the bottom end 29 so as to extend outward along a central longitudinal axis X of rotation of the set screw 1. The set screw 1 in FIG. 1 exerts a compression or clamping force onto the elongated member 3 through the bottom tip 28. The bottom tip 28 impinges on the elongated member 3 and may form a dimple or depression in the elongated member 3. Bottom tip 28 is substantially dome shaped with a surface that is convex or rounded so that a small surface area is in contact with the elongated member 3 providing a strong grip when pressed against elongated member 3. Alternatively, the bottom tip 28 could be of various shaped points including flat, cone, cup, dog, or knurled.

Referring now to FIG. 5, a hollow bore 44 is formed in the body of the set screw 1 and the hollow bore 44 extends from an opening in the upper portion 40 through the frangible portion 32 and into at least a portion of the lower portion 42. The portion of the hollow bore 44 extending into the lower portion 42 comprises a driving recess 45 or socket for assisting in the removal by internal wrenching of the lower portion 42 after the upper portion 40 has been broken away at the frangible portion 32. The driving recess 45 is a recess and has a shape that can be a hexagon, hexalobular, clutch, fluted, frearson, slotted, star, Torx, reverse thread, Pozidriv, or Phillips. The frangible portion 32 is an area of reduced wall thickness and can have a cross-section in the form of a notch creating a weakened zone. The upper portion 40 can have at least one flat surface 49 for the application of torque to the set screw 1. This flat surface 49 can be on the exterior of the upper portion 40.

A frangible portion 32 connects the upper portion 40 and the lower portion 42 of the set screw 1. The frangible portion 32 is easily or readily ruptured, separated or broken when a pre-selected torque or force is applied. When the upper portion 40 is removed, the lower portion 42 of the set screw 1 remains as shown in FIG. 6. An exemplary description of a set screw 1 having such a frangible portion 32 is disclosed in U.S. Pat. No. 6,179,841 and this patent is incorporated herein by reference in its entirety.

The present invention also comprises an external thread 5 on the outer surface of the lower portion 42 wherein at least a portion of the external thread 5 has a variable pitch. The variable pitch provides the spinal implant system 2 with a press fit threaded engagement which prevents application of an excessive clamping force on the elongated member 3 even when an excessive amount of torque is applied during installation of the set screw 1.

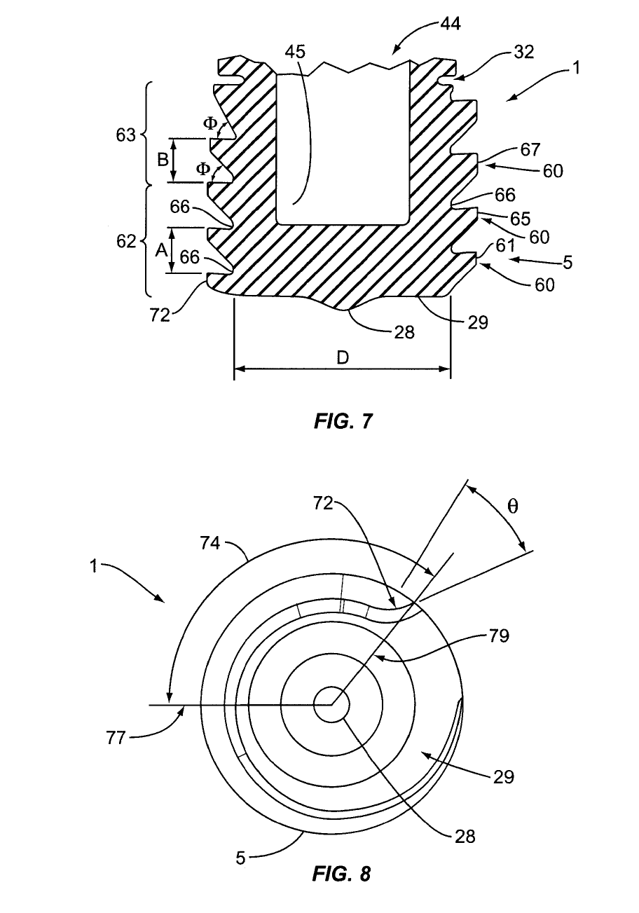

In FIG. 7 an external thread 5 is illustrated. Pitch, as used herein, is the longitudinal distance from a point on one ridge 60 to the corresponding point on the adjacent ridge 60 of the external thread 5. Variable pitch, as used herein, refers to the pitch varying along the axial length of the set screw 1 (dimension A is not equal to dimension B). If the pitch did not vary and was a standard pitch, the pitch would equate to the distance per ridge 60 of thread. In the present invention, the distance per ridge 60 of thread may be one value in a first portion 62 of the external thread 5 and another value in a second portion 63 of the external thread 5. This external thread 5 has a single start and is a single continuous thread. Pitch includes the concept of when the distance from the crest 61, 65, 67 of at least one ridge 60 to at least one of its corresponding roots 66 at the root diameter D is compared to the corresponding distance from the crest 61, 65, 67 of an adjacent ridge 60 to at least one of its corresponding roots 66. As the pitch changes or varies, the thread angle Φ may also vary. This variation in thread angle Φ may also be representative of a thread having a variable pitch.

The cross-sectional shape of the external thread 5 is illustrated as a buttress thread form which gives the appearance of a truncated scalene triangle. It is appreciated that various other thread forms may be used in the present invention including square, trapezoidal, or other triangular shapes such as an isosceles triangle. The thread angle is characteristic of the cross-sectional shape selected. While a 60° thread angle may be common, the external thread 5 illustrated preferably has about a 45° reverse angle buttress thread form. The external thread 5 includes at least a portion of the external thread 5 having a standard pitch and at least a portion having a variable pitch. The external thread 5 has at least a first crest 61 and second crest 65. Preferably the variable pitch of the external thread 5 starts after the second crest 65. The external thread 5 may alternatively have a variable pitch along the entire length of the external thread 5.

The external thread 5 not only has a variable pitch but also includes a truncation cut 74 at the thread start 72. Referring to FIG. 8, the external thread 5 includes a thread start 72 at the bottom end 29 of the set screw 1. Thread start 72, as used herein, means the location at the bottom end 29 of the set screw 1 where the external thread 5 begins. The thread start 72 has a truncation cut 74 so the threading begins smoothly and consistently at the same location. A beginning of the truncation cut 77 is preferably at an angle of greater than 120° from an exit of the truncation cut 79. A reference datum is located on the set screw 1 and can be located on the upper portion 40 or elsewhere. The truncation cut 74 may start at a location aligned with or relative to the reference datum. The truncation cut 74 is timed or registered such that the thread start 72 of the external thread 5 consistently enters the internal thread 7 of the threaded opening 11 at a particular circumferential location with respect to the U-shaped receptacle 21 of the receiver. The truncation cut 74 begins at a particular location so that the set screw 1 will engage the receiver member 20 by a predetermined longitudinal distance before encountering the variable pitch on the external thread 5 of the set screw 1. At the exit of the truncation cut 79 there is a truncation exit angle θ that is preferably about 35°.

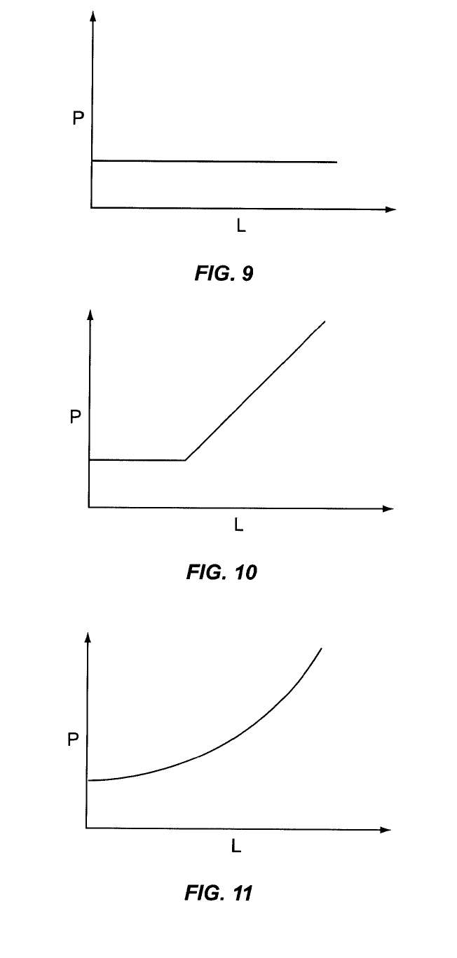

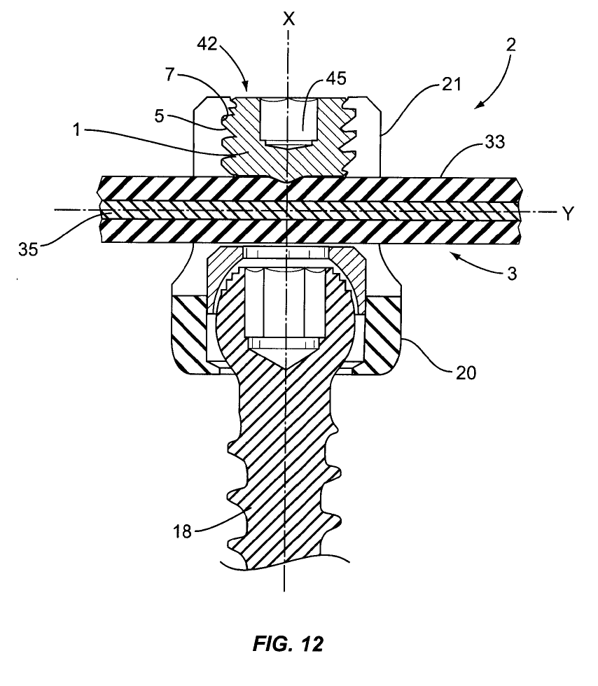

A plot of pitch “P” versus axial length “L”, where the axial length corresponding to the particular pitch is measured longitudinally from the bottom end 29 of the set screw 1 toward the upper portion 40, for a variety of pitch configurations is illustrated in FIGS. 9-11. In particular, FIG. 9 illustrates a standard pitch that is constant. FIG. 10 illustrates a variable pitch wherein the initial pitch is a standard pitch and after a specified axial length there is a constant increase in the pitch. FIG. 11 illustrates a variable pitch configuration wherein the pitch increases very slightly in an initial distance and then the pitch increases as the axial length increases. The slope of the line changes relative to the P axis as the variable pitch is encountered.

The external thread 5 in accord with the present invention can be generated in numerous ways. The external thread 5 having a variable pitch may be cut into the lower portion 42 with a lathe, tap, die, or milled using a milling machine having a rotating cutter moving in a helical path. The rotational speed and linear advancement rate at which the thread milling occurs could create an external thread 5 having a variable pitch. Alternatively the variable pitch in the external thread 5 may be created by a thread rolling machine.

The external thread 5 of the lower portion 42 of the set screw 1 can be referred to as male and the internal thread 7 of the threaded opening 11 of the U-shaped receptacle 21 of the receiver can be referred to as female. Mating male and female pitch diameters are determined based on the desired fit between the male and female threads. Typically, male and female threads will properly mate when their sloping faces are in contact. This contact between sloping faces typically happens when the pitch diameters of male and female threads match closely.

In a traditional set screw configuration, the set screw 1 is rotatably installed into the threaded opening 11 and progresses through the threaded opening 11 until the set screw 1 abuts against another item such as an elongated member 3 that is inserted into the U-shaped receptacle 21. When the set screw 1 contacts the elongated member 3 the torque required to progress further into the threaded opening 11 increases. Continued tightening of the set screw 1 produces a clamping force by the set screw 1 against the elongated member 3. The torque used when tightening the set screw 1 against the elongated member 3 to secure the elongated member 3 in the U-shaped receptacle 21 is proportionally related to the clamping force applied against the elongated member 3. When the external thread 5 of the set screw 1 has a variable pitch, the clamping force applied against the elongated member 3 is not proportionally related to the torque that is used to tighten the set screw 1. This relationship is no longer proportional because the variable pitch of the external thread 5 on the set screw 1 is press fit against the internal thread 7 of the threaded opening 11 while securing the elongated member 3.

The external thread 5 is arranged to engage with the internal thread 7 of the threaded opening 11 in such a manner as to allow the set screw 1 to travel a predetermined distance into the receiver prior to engaging the variable pitch portion of the set screw 1. In one exemplary configuration, an M8 sized set screw 1 with a standard pitch that is about 1.25 mm could be used having a variable pitch that is greater than about 1.25 mm, and preferably the variable pitch is between about 1.25 mm to about 2 mm, and even more preferably the variable pitch is less than about 3 mm. In a more preferable embodiment, the pitch of the external thread 5 begins at a standard pitch of about 1.25 mm and ramps or transitions up to a pitch of about 2 mm over an axial length of about 1.6 mm. The thread angle in this variable pitch region is preferably about 45° or less.

As the external thread 5 and the internal thread 7 threadably engage with each other, the variable pitch portion of the external thread 5 is reached when the pitch diameter of the external thread 5 and the internal thread 7 no longer match closely. At the location where the variable pitch of the external thread 5 deviates or sufficiently varies from the pitch of the internal thread 7, the axial movement of the set screw 1 into the receiver member 20 is curtailed and the torque necessary for continuing to engage the set screw 1 into the receiver member 20 increases. The longitudinal distance traveled before the internal thread 7 of the threaded opening 11 of the U-shaped receptacle 21 engage the variable pitch portion of the set screw 1 or becomes seated in the variable pitch portion is at least greater than about 2 mm, and is preferably between about 2 mm to about 3 mm, and even more preferably is about 2.375 mm. This is the point at which the set screw 1 is deemed to have become seated within the receiver member 20 and is the location at which the preselected torque value is reached. The preselected torque value is preferably chosen such that at least about two ridges 60 of the external thread 5 engage the internal thread 7 before the variable thread portion is engaged. This preselected torque value is preferably between about 4 Nm to about 8 Nm.

It is notable that the preselected torque value for seating the set screw 1 is selected at a value less than the break-off or separation torque required to cause the frangible portion 32 to snap or break away separating the upper portion 40 from the lower portion 42 of the set screw 1. The break-off torque for the frangible portion 32 is a predetermined amount greater than the torque required to seat the set screw 1. The break-off torque of the frangible portion 32 is preferably between about 8 Nm to about 12.5 Nm.

The elongated member 3 for use in the spinal implant system 2 may be constructed from a variety of surgical grade materials, and may be in whole or in part constructed of biocompatible materials of various types including metals or polymers. For example, the elongated member 3 can be constructed of the following biocompatible materials, but are not limited to, non-cobalt-chromium alloys, titanium alloys, nickel titanium alloys, and/or stainless steel alloys, reinforced composite materials, plastics and polymers including without limitation any member of the polyaryletherketone (PAEK) family such as polyetheretherketone (PEEK), polyether ketone (PEK), carbon fiber reinforced PEEK (CFRP), or polyetherketoneketone (PEKK); polysulfone; polyetherimide; polyimide; ultra-high molecular weight polyethylene (UHMWPE); and/or cross-linked UHMWPE. The elongated member 3 may be straight, curved, or comprise one or more curved portions along its length. An exemplary elongated member 3 for use with the present invention is the polymeric pedicle rod disclosed in U.S. patent application Ser. No. 12/234,091 which corresponds to publication No. US 2009/0163955 A1.

The preferred spinal implant system 2 includes an elongated member 3, a set screw 1 and a receiver member 20. The elongated member 3 can be a metal rod or can have an outer surface 33 that is polymeric with an inner core 35 of CFRP. The set screw 1 includes an upper portion 40 connected to a lower portion 42 by a frangible portion 32. The lower portion 42 is separable from the upper portion 40 by breaking the frangible portion 32. The lower portion 42 has an external thread 5 with a variable pitch. The receiver member 20 has a proximal end 39 and a distal end 37. The distal end 37 can be a bone anchor 18 that is adapted to be affixed to the spinal column of a patient during spine surgery. The proximal end 39 of the receiver member 20 has a threaded opening 11 leading to a U-shaped receptacle 21 between a first extending arm 22 and a second extending arm 23. The threaded opening 11 is configured to engage the external thread 5 of the set screw 1. The elongated member 3 is capable of being secured within the U-shaped receptacle 21 of the receiver member 20 by the set screw 1 without causing detrimental deformation of the polymeric outer surface 33 of the elongated member 3. The preselected torque for seating the set screw 1 occurs at a predetermined distance of longitudinal engagement between the set screw 1 and the receiver member 20 in order to provide the desired pre-tension or clamping force on the elongated member 3 by the bottom tip 28 of the set screw 1 to capture and secure the elongated member 3 in the U-shaped receptacle 21 of the receiver member 20. The break-off torque for separation of the frangible portion 32 is greater than the amount of preselected torque needed to secure the elongated member 3 in the U-shaped receptacle 21 of the receiver member 20 by the set screw 1. Since the set screw 1 has become seated in the threaded opening 11 further increasing the torque to its break-off torque value is achieved without increasing the clamping force or further significantly compressing or tightening of the elongated member 3. This allows the proper clamping force to be maintained and the break-off torque to be reached in order for the frangible portion 32 to separate the upper portion 40 from the lower portion 42 without damaging the elongated member 3.

In use, the set screw 1 may be inserted in the threaded opening 11 of the U-shaped receptacle 21 after the receiver member 20 is affixed to a bone in the spinal column of a patient and after an elongated member 3 is inserted through the U-shaped receptacle 21. The set screw 1 is tightened to secure the elongated member 3 in position, thereby preventing further rotational or translational movement of the elongated member 3 with respect to the U-shaped receptacle 21, the set screw 1 is further tightened until the preselected torque is achieved, the set screw 1 is seated in the receiver member 20, and the set screw 1 will have traveled the appropriate axial length for the bottom tip 28 of the set screw 1 to engage and clamp onto the elongated member 3.

Further driving or tightening of the set screw 1 while the set screw 1 is seated produces a torque on the set screw 1 equal to the break-off torque and causes the upper portion 40 of the set screw 1 to shear off from the lower portion 42 along the frangible portion 32. The lower portion 42 of the set screw 1 remains seated in the internal thread 7 of the threaded opening 11 of the receiver member 20 and remains able to secure the elongated member 3 and prevent relative movement of the elongated member 3 with respect to the receiver member 20 even under substantial load. Consequently, the external thread 5 with variable pitch provides a consistent clamping force even when excessive torque is applied to the set screw 1.

While the set screw 1 has been described including an upper portion 40 and a lower portion 42, as shown in FIG. 3 a set screw 1 having an external thread 5 with a variable pitch and a truncation cut 74 may alternatively be configured only having a lower portion 42, as shown in FIG. 6. In such a configuration, the lower portion 42 can be installed into the threaded opening 11 of the receiver member 20 through use of an instrument or tool by use of the driving recess 45.

While the present invention has been illustrated by the above description of embodiments, and while the embodiments have been described in some detail including exemplary dimensional values as they relate to a particular M8 sized set screw 1, it is not the intention of the applicant to restrict or in any way limit the scope of the invention to such detail. Additional advantages and modifications will readily appear to those skilled in the art. Therefore, the invention in its broader aspects is not limited to the specific details, representative apparatus and methods, and illustrative examples shown and described. Accordingly, departures may be made from such details without departing from the spirit or scope of the applicant's general or inventive concept.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise. It is understood that all spatial references, such as “longitudinal axis,” “horizontal,” “vertical,” “top,” “under”, “below”, “lower”, “over”, “upper”, “bottom,” “left,” “right,” and the like, are for illustrative purposes only and can be varied within the scope of the disclosure. Further, terms such as “first”, “second”, and the like, are also used to describe various elements, regions, sections, etc and are also not intended to be limiting. Like terms refer to like elements throughout the description.

What is claimed is:

1. A set screw for use with a receiver to secure an elongated member in a spinal implant system, the set screw comprising:
a body having a lower portion;
an external thread on the lower portion wherein at least a portion of the external thread has a variable pitch, and wherein the external thread of the set screw includes a thread start having a truncation cut.

2. The set screw according to claim 1, wherein the truncation cut begins at a location aligned with a reference datum on the set screw so that the set screw will engage the receiver member by a predetermined distance before encountering the variable pitch of the set screw.

3. The set screw according to claim 1, wherein the external thread includes at least a portion of the external thread having a standard pitch

4. The set screw according to claim 1, wherein the external thread is a single continuous thread.

5. The set screw according to claim 1, wherein the external thread is a reverse angle thread.

6. The set screw according to claim 1, wherein the lower portion includes a driving recess.

7. The set screw according to claim 6, wherein the driving recess is a recess having a shape selected from the group consisting of hexagon, hexalobular, clutch, fluted, frearson, slotted, star, Torx, reverse thread, Pozidriv, or Phillips.

8. A set screw for use with a receiver to secure an elongated member in a spinal implant system, the set screw comprising:
a body having an upper portion and a lower portion, the upper portion being connected to the lower portion through a frangible portion;
an external thread on the lower portion wherein at least a portion of the external thread has a variable pitch.

9. The set screw according to claim 8, wherein the external thread includes at least a portion of the external thread having a standard pitch and at least a portion having a variable pitch.

10. The set screw according to claim 8, further comprising at least a first and second crest of the external thread, and wherein the external thread has a variable pitch that starts after the second crest.

11. The set screw according to claim 8, wherein the external thread is a reverse angle thread.

12. The set screw according to claim 8, wherein the external thread includes a thread start having a truncation cut.

13. The set screw according to claim 12, further comprising a beginning of the truncation cut at an angle of greater than 120° from an exit of the truncation cut.

14. The set screw according to claim 13, further comprising a reference datum on the upper portion, and wherein the truncation cut begins at a location aligned with the reference datum.

15. The set screw according to claim 8, wherein the external thread has a variable pitch along the entire length of the thread.

16. A spinal implant system comprising;
an elongated member having at least an outer surface; and
a set screw including an upper portion connected to a lower portion by a frangible portion, the lower portion separable from the upper portion by breaking at the frangible portion, the lower portion having an external thread with a variable pitch; and
a receiver member having a proximal end and a distal end, the distal end adapted to be affixed to the vertebral column of a patient, the proximal end having a threaded opening leading to a receptacle, wherein the threaded opening is configured to engage the external thread of the set screw; and
wherein the elongated member is capable of being secured within the receptacle of the receiver member by the set screw without causing detrimental deformation of the outer surface of the elongated member and wherein a break-off torque for separation of the frangible portion is greater than the amount of torque needed to secure the elongated member in the receptacle of the receiver member by the set screw.

17. The spinal implant system according to claim 16, further comprising a first extending arm and a second extending arm forming the receptacle and, wherein the first extending arm has a first proximal face and the second extending arm has a second proximal face, and the first proximal face has at least a partial thread formed therein to engage the exterior thread of the set screw.

18. The spinal implant system according to claim 17, wherein the second proximal face has wholly omitted any thread being formed therein to assure the external threads engage the first extending arm before engaging the second extending arm in order to avoid cross-threading of the exterior thread on the set screw.

19. The spinal implant system according to claim 18, wherein the external thread of the set screw includes at least a portion of the external thread having a standard pitch and at least another portion having a variable pitch.

20. The spinal implant system according to claim 19, wherein the external thread of the set screw includes a thread start having a truncation cut, and wherein the truncation cut begins at a location aligned with a reference datum on the upper portion of the set screw so that the set screw will engage the receiver member by a predetermined distance before encountering the variable pitch of the set screw.
